(19) United States
(12) Patent Application Publication
(10) Pub. No.: US 2005/0272936 A1
(43) Pub. Date: Dec. 8, 2005

(54) Imidazolium cxcr3 inhibitors

(76) Inventors: Jeffrey M. Axten, Collegeville, PA (US); James J. Foley, King of Prussia, PA (US); William D. Kingsbury, Collegeville, PA (US); Henry M. Sarau, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/516,970

(22) PCT Filed: May 29, 2003
(86) PCT No.: PCT/US03/16782

§ 371 Date: Dec. 3, 2004

Related U.S. Application Data
(60) Provisional application No. 60/385,658, filed on Jun. 3, 2002.

(51) Int. Cl. C07D 233/54; C07D 43/02

(52) U.S. Cl. 548/311.1; 548/335.1; 548/341.1

(57) ABSTRACT

Imidazolium compounds of formula I compound of formula (I) 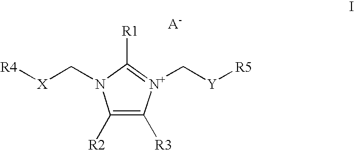 wherein:
A⁻ is an anion;
R₁, R₂ and R₃ are the same or different and are hydrogen, C₁-C₆ alkyl, aryl, fused aryl, or heteroaryl; or are substituted aryl, fused aryl or heteroaryl;
X and Y are the same or different and are —CH₂, —C═O, —CHOH, —C═S, or —C═NR₆;
R₄ and R₅ are the same or different and are aryl, substituted aryl, heteroaryl, or substituted heteroaryl; and
R₆ is aryl, C₁-C₆ alkyl, OH, C₁-C₆ alkoxy, or aryloxy. are useful as inhibitors of a chemokine receptor knows as CXCR3 and its equivalent chemokine receptors.

In general this invention relates to certain imidazoliums and their use as inhibitors of a chemokine receptor knows as CXCR3 and its equivalent chemokine receptors.

### BACKGROUND OF THE INVENTION

Chemokines are chemotactic cytokines that are released by a variety of cells to attract macrophages, T cells, eoxinophils, basophils and neutrophils to sites of inflammation [see Schall, Cytokine, 3:165-183 (1991), Schall, et al, Curr. Opin. Immunol., 6:865-873 (1994) and Murphy, Rev. Immun., 12:593-663 (1994)]. In addition to stimulating chemotaxis other changes can be selectively induced by chemokines in responsive cells including changes in cell shape, transient rises in the concentration of intracellular free calcium ions, granule exocytosis, integrin upregulation, formation of bioactive lipids and respiratory bursts associated with leukocyte activation. Thus chemokines are early triggers of the inflammatory response, causing inflammatory mediator release, chemotaxis and estravasation to the sites of infection or inflammation.

There are four classes of chemokines, CXC (α), CC (β), C(γ), and CX₃C (δ), depending on whether the first two cysteines are separated by a single amino acid (C—X—C), are adjacent (C—C), have a missing cysteine pair (C), or are separated by three amino acides (CXC₃). The α-chemokines, such as interluken-8 (IL-8), melanoma growth stimulatory activity protein (MGSA), and stromal cell derived factor 1 (SDF-1) are chemotactic primarily for neutrophils and lymphocytes, whereas β-chemokines, such as RANTES, MIP-1α, MIP-1β, monocyte chemotactic protein-1 (MCP-1), MCP-2, MCP-3 and eotaxin are chemotactic for macrophages, T-cells, eosinophils and basophils (Deng, et al., Nature, 381:661-666 (1996)). The C chemokine lymphotactin shows specificity for lymphocytes (Kelner, et al., Science, 266:1395-1399 (1994)) while the CX₃C chemokine fractalkine shows specificity for lymphocytes and monocytes (Bazan, et al., Nautre, 385:640-644 (1997)).

The chemokines bind specific cell-surface receptors belonging to the family of G-protein-coupled seven-transmembrane-domain proteins (reviewed in Horuk, Trends Pharm. Sci., 15,159-165 (1994)) which are termed “chemokine receptors.” On binding their cognate ligands, chemokine receptors transduce an intracellular signal though the associated trimeric G protein, resulting in a rapid increase in intracellular calcium concentration. There are at least seven human chemokine receptors that bind or respond to .beta.-chemokines with the following characteristic pattern: CCR-1 (or “CKR-1” or “CC-CKR-1”) [MIP-1.alpha., MIP-1.beta., MCP-3, RANTES] (Ben-Barruch, et al., J. Biol. Chem., 270, 22123-22128 (1995); Beote, et al, Cell, 72, 415425 (1993)); CCR-2A and CCR-2B (or “CKR-2A”/“CKR-2A” or “CC-CKR-2A”/“CC-CKR-2A”) [MCP-1, MCP-3, MCP-4]; CCR-3 (or “CKR-3” or “CC-CKR-3”) [eotaxin, RANIES, MCP-3] (Combadiere, et al., J. Biol. Chem., 270, 16491-16494 (1995); CCR-4 (or “CKR-4” or “CC-CKR-4”) [MIP-1.alpha., RANTES, MCP-1] (Power, et al., J. Biol. Chem., 270, 19495-19500 (1995)); CCR-5 (or “CKR-5” or “CC-CKR-5”) [MIP-1.alpha., RANTES, MIP-1.beta.] (Sanson, et al., Biochemistry, 35, 3362-3367 (1996)); and the Duffy blood-group antigen [RANTES, MCP-1] (Chaudhun, et al., J. Biol. Chem., 269, 7835-7838 (1994)). The β-chemokines include eotaxin, MIP (“macrophage inflammatory protein”), MCP (“monocyte chemoattractant protein”) and RANTES (“regulation-upon-activation, normal T expressed and secreted”).

Chemokine receptors, such as CCR1, CCR2, CCR2A, CCR2B, CCR3, CCR4, CCR5, CCR6, CCR7, CCR8, CCR9, CXCR1, CXCR2, CXCR3, CXCR4, CXCR5, CX₃CR1, and XCR1 have been implicated as being important mediators of inflammatory and immunoregulatory disorders and diseases, including asthma and allergic diseases, as well as autoimmune pathologies such as rheumatoid arthritis and atherosclerosis.

Chemokine receptors such as CXCR-3 have been implicated as being important mediators of inflammatory and immunoregulatory disorders and diseases, including asthma and allergic diseases, as well as autoimmune pathologies such as rheumatoid arthritis and atherosclerosis. For example it plays a pivotal role in attracting eosinophils to sites of allergic inflammation. Accordingly, agents, which modulate it, would be useful in such disorders and diseases. The compounds of this invention are inhibitors of this chemokine receptor and as such are useful in treating diseases which the CXCR3 chemokine receptor is involved

### SUMMARY OF THE INVENTION

In a first aspect this invention relates to a compound of formula (I) 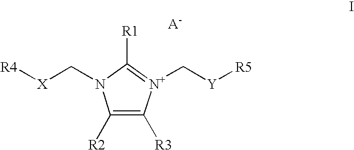
wherein:
A⁻ is an anion;
R₁, R₂ and R₃ are the same or different and are hydrogen, C₁-C₆ alkyl, aryl, fused aryl, or heteroaryl; or are substituted aryl, fused aryl or heteroaryl;
X and Y are the same or different and are —CH₂, —C═O, —CHOH, —C═S, or —C═NR₆;
R₄ and R₅ are the same or different and are aryl, substituted aryl, heteroaryl, or substituted heteroaryl; and
R₆ is aryl, C₁-C₆ alkyl, OH, C₁-C₆ alkoxy, or aryloxy.

In other respects, this invention relates to pharmaceutically acceptable preparations containing one or more of the compounds of formula (I) or a hydrate thereof. Uses of these compositions for treating diseases in which the CXCR3 receptor is involved are within the scope of this invention. Methods for preparing the compounds of formula (I) are also within the scope of this invention.

### DESCRIPTION OF THE INVENTION

The term “alkyl” means a straight or branched chain radical that may be fully saturated or mono- or polyunsaturated and can include di- and multivalent radicals. Examples of saturated hydrocarbon radicals include methyl, ethyl, n-propyl, isopropyl, n-butyl, t-butyl, isobutyl, sec-butyl, n-pentyl, and n-hexyl and the like. An unsaturated alkyl group has one or more double bonds or triple bonds. Examples of unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “aryl” mean a polyunsaturated aromatic group which can be a single ring or multiple rings which are fused (“fused aryl”) together or linked covalently. The term “heteroaryl” refers to aryl groups that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazoly, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquionolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

Preferred aryl groups are phenyl and substituted phenyl. Preferred fused aryl groups are naphthyl and phenanthrenyl. Preferred substituted aryls include phenyl and naphthyl.

The terms “alkoxy” and “aryloxy” refer to those groups attached to the remainder of the molecule via an oxygen atom.

Substituents for the aryl, fused aryl, and heteroaryl groups are: -halogen, —OR′, —OC(O)R′, —NR′R′″, —SR′, —R′, —CN, —NO₂, —CO₂R′, —CONR′R′″, —C(O)R′, —OC(O)NR′R″, —NR″ C(O)R′, —NR″ C(O)₂R′, —NR′—C(O)NR′ R′″, —NH—C(NH₂)═NH, —NR′C(NH₂)═NH, —NH—C(NH₂)—NR′, —S(O)R′, —S(O)₂R′, —S(O)₂NR′R″, —N₃, —CH(Ph)₂, perfluoro(C₁-C₄)alkoxy, and perfluoro(C₁-C₄)alkyl. The number of such substituents can range from one to some number equal to the reactable atoms in the aromatic ring. In the foregoing groups, R′, R″ and R′″ are independently selected from hydrogen, (C₁-C₈)alkyl, unsubstituted aryl and heteroaryl, (unsubstituted aryl)-(C₁-C₄)alkyl, and (unsubstituted aryl)oxy-(C₁-C₄)alkyl.

Anions may be any negatively charged, pharmaceutically acceptable group. The halides are preferred, particularly the bromide ion.

Preferred compounds are those wherein R₁ is hydrogen or methyl; R₄ and R₅ are a phenyl group substituted preferably at the 3, and 4-positions by two Z groups; X and Y are —C═O; and Z is methyl or CF₃. And one or more of the compounds set out in the Examples below may be preferred.

Diseases or conditions of humans or other species which can be treated with inhibitors of chemokine receptor function include: inflammatory or allergic diseases and conditions, including respiratory allergic diseases such as asthma, allergic rhinitis, hypersensitivity lung diseases, hypersensitivity pneumonitis, eosinophilic pneumonias (e.g., Loeffler's syndrome, chronic eosinophilic pneumonia), delayed-type hypersensitivity, interstitial lung diseases (ILD) (e.g., idiopathic pulmonary fibrosis, or ILD associated with rheumatoid arthritis, systemic lupus erythematosus, ankylosing spondylitis, systemic sclerosis, Sjogren's syndrome, polymyositis or dermatomyositis); systemic anaphylaxis or hypersensitivity responses, drug allergies (e.g., to penicillin, cephalosporins), insect sting allergies; autoimmune diseases, such as rheumatoid arthritis, psoriatic arthritis, multiple sclerosis, systemic lupus erythematosus, myasthenia gravis, juvenile onset diabetes; glomerulonephritis, autoimmune thyroiditis, Behcet's disease; graft rejection (e.g., in transplantation), including allograft rejection or graft-versus-host disease; inflammatory bowel diseases, such as Crohn's disease and ulcerative colitis; spondyloarthropathies; scleroderma; psoriasis (including T-cell mediated psoriasis) and inflammatory dermatoses such an dermatitis, eczema, atopic dermatitis, allergic contact dermatitis, urticaria; vasculitis (e.g., necrotizing, cutaneous, and hypersensitivity vasculitis); eosinphilic myositis, eosinophilic fasciitis; cancers with leukocyte infiltration of the skin or organs. Other diseases or conditions in which undesirable inflammatory responses are to be inhibited can be treated, including, but not limited to, reperfusion injury, atherosclerosis, certain hematologic malignancies, cytokine-induced toxicity (e.g., septic shock, endotoxic shock), polymyositis, dermatomyositis.

The pharmaceutical compositions for the administration of the compounds of this invention may conveniently be presented in dosage unit form and may be prepared by any of the methods well known in the art of pharmacy. All methods include the step of bringing the active ingredient into association with the carrier that constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

Inhibitors of CXCR3 can be administered by injection in solutions either intravenously, intramuscularly, intraperitoneally, or orally. These doses will contain the drug in the range of 1 to 140 mgkg of body weight. The solution preferably contains a buffer (such as phosphate) to keep the pH in the range of about 3.5 to 7. DMSO or alcoholic solvents may also be present (at concentrations such as 0.01 to 10 mL/liter) to aid solubility.

No untoward effects are expected if the invention is practiced in form and spirit with the description provided herein.

Compound Synthesis

Exemplary bisphenacylimidazolium salts of this invention, such as 3 in Scheme 1 below, may be prepared by reacting an imidazole such as 1-Scheme 1 with an excess molar amount of a phenacyl halide such as 2-Scheme 1 in a polar organic solvent such as dimethylformamide or acetonitrile for example. The major product obtained is the dialkylated imidazolium 3.

Alternatively, using and excess molar of the imidazole 1 will provide the monoalkyl product such as 4-Scheme 1 as the major product. Reaction of 4 with an additional aquivalent of a phenacylhalide such as 5-Scheme 1 affords a non-symmetrical imidazolium such as 6-Scheme 1.

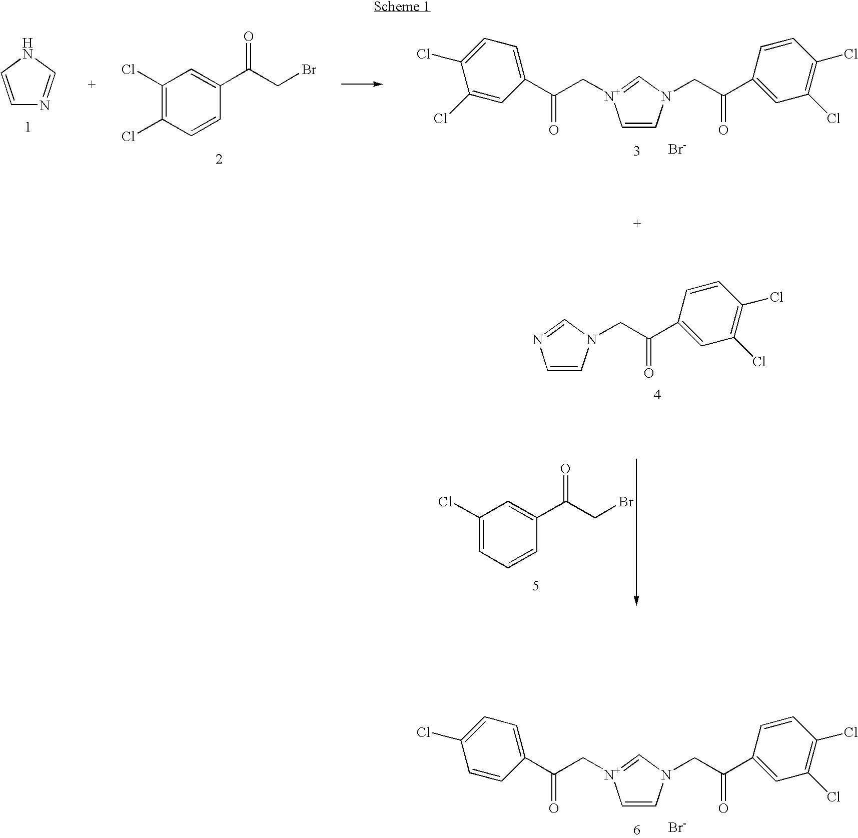

### EXAMPLES

### Example 1

### Preparation of 3-Benzyl-1-[2-(3,4-dichlorophenyl)-2-oxoethyl]-2-methyl-3H-imidazol-1-ium; bromide

1-Benzyl-2-methyl-1H-imidazole (50 mg, 0.29 mmol) and 3,4-dichlorophenacyl bromide (86 mg, 0.32 mmol) were stirred in 0.2 ml of acetonitrile (a few drops of dimethylformamide were added for solubility if necessary) overnight at room temperature. The mixture was diluted with ether, the solid product filtered, and washed with ether and dried to give 119 mg of product. LC/MS (ES+) m/e 361[M+1]

### Example 2

### Preparation of 3-[2-(3,4-Difluorophenyl)-2-oxoethyl]-1-[2-(3-fluoro-phenyl)-2-oxoethyl]-3H-imidazol-1-ium; bromide

1-(3,4-Difluorophenyl)-2-imidazol-1-yl-ethanone (50 mg, 0.228 mmol) and 2-bromo-1-(3-fluorophenyl)-ethanone (54 mg, 0.249 mmol) were stirred in 0.2 ml of acetonitrile (a few drops of dimethylformamide were added for solubility) overnight at room temperature. The mixture was diluted with ether, the solid filtered, washed with ether and dried to provide 87 mg of product. LC/MS (ES+) m/e 360[M+1]

### Example 3

### Preparation of 3-[2-(3,4-Difluorophenyl)-2-oxoethyl]11-(2-oxo-2-phenylethyl)-3H-imidazol-1-ium; bromide

Following the procedure in Example 2 except substituting with 2-bromoacetophenone. 84 mg of captioned product was obtained. LC/MS (ES+) m/e 342[M+1]

### Example 4

### Preparation of 3-[2-(3,4-Difluorophenyl)-2-oxoethyl]-1-[2-(3-methoxyphenyl)-2-oxoethyl]-3H-imidazol-1-ium; bromide

The caption compound was made following the procedure in Example 2, except substituting 2-bromide-1-(3-fluorophenyl)-ethanone with 2-bromo-1-(3-methoxyphenyl)-ethanone. 90 mg of captioned product was obtained. LC/MS (ES+) m/e 372[M+1]

### Example 5

### Preparation of 1,3-Bis-[2-(3,4-difluorophenyl)-2-oxoethyl]-3H-imidazol-1-ium; bromide

Following the procedure in Example 2 except substituting 2-bromo-1-(3-fluoro-phenyl)-ethanone with 2-bromo-1-(3,4-difluorophenyl)-2-imidazol-1-yl-ethanone. 97 mg of the caption product was obtained. LC/MS (ES+) m/e 378[M+1]

### Example 6

### Preparation of 1-[2-(3,4-Dichlorophenyl)-2-oxoethyl]-3-[2-(3,4-difluorophenyl)-2-oxoethyl]-3H-imidazol-1-ium; bromide

Following the procedure in Example 2 except substituting 2-bromo-1-(3-fluorophenyl)-ethanone with 2-bromo-1-(3,4-dichlorophenyl)-2-imidazol-1-yl-ethanone. 98 mg of product was obtained. LC/MS (ES+) m/e 411[M+1]

### Example 7

### Preparation of 3-[2-(3,4-Dichlorophenyl)-2-oxoethyl]-1-phenethyl-3H-imidazol-1-ium; bromide

Following the procedure in Example 2 except substituting starting materials with 3,4-dichlorophenacyl bromide and 1-phenethyl-1H-imidazole. 59 mg of product was obtained. LC/MS (ES+) m/e 361[M+1]

### Example 8

### Preparation of 1,3-Bis-[2-(3-fluorophenyl)-2-oxoethyl]-3H-imidazolium; bromide

1-(3-Fluorophenyl)-2-imidazol-1-yl-ethanone (46 mg 0.228 mmol) and 3-fluoro-phenacyl bromide (58 mg, 0.248 mmol) were stirred in 0.2 ml of acetonitrile overnight at room temperature. The mixture was diluted with ether, the solid filtered, washed with ether and dried to give 85 mg of the captioned product. LC/MS (ES+) m/e 342[M+1]

### Example 9

### Preparation of 3-[2-(3-Fluorophenyl)-2-oxoethyl]-1-[2-(3-methoxy-phenyl)-2-oxoethyl]-3H-imidazolium; bromide

Following the procedure of Example 8 except substituting 3-fluorophenacyl bromide with 2-bromo-1-(3-methoxy-phenyl)-ethanone. 52 mg of product was obtained. LC/MS (ES+) m/e 354 [M+1]

### Example 10

### Preparation of 3-[2-(3-Fluorophenyl)-2-oxoethyl]-1-[2-oxo-2-phenyl-ethyl]-3H-imidazolium; bromide

Following the procedure in Example 8, except substituting 3-fluorophenacyl bromide with phenacyl bromide 72 mg of the captioned product was obtained. LC/MS (ES+) m/e 324[M+1]

### Example 11

### Preparation of 1-[2-(3,4-Dichlorophenyl)-2-oxoethyl]-3-[2-(3-fluoro-phenyl)-2-oxoethyl]-3H-imidazolium; bromide

Following the procedure in Example 8 except replacing 3-fluorophenacyl bromide with 3,4-dichlorophenacyl bromide. 92 mg of product was obtained. LC/MS (ES+) m/e 393[M+1]

### Example 12

### Prepartion of 1-[2-(3-Chlorophenyl)-2-oxoethyl]-3-[2-(3-fluoro-phenyl)-2-oxoethyl]-3H-imidazol-ium; bromide

Following the procedure in Example 8 except subsitituting the starting materials with 3-chloro-phenacyl bromide and 1-(3-fluoro-phenyl)-2-imidazol-1-yl-ethanone. 75 mg of the captioned product was obtained. LC/MS (ES+) m/e 359[+1]

### Example 13

### Preparation of 1-[2-(3-Chlorophenyl)-2-oxoethyl]-3-[2-(3,4-difluorophenyl)-2-oxoethyl]-3H-imidazolium; bromide

Following the procedure in Example 8 except subsitituting the starting materials with 3-chlorophenacyl bromide and 1-(3,4-difluorophenyl)-2-imidazol-1-yl-ethanone. 77 mg of the captioned product was obtained. LC/MS (ES+) m/e 377[M+1]

### Example 14

### Preparation of 3-Benzyl-1-(3,4-dichlorobenyl)-2-methyl-3H-imidazol-1-ium; bromide

1-Benzyl-2-methyl-1H-imidazole (50 mg, 0.3 mmol) and 3,4-dichlorobenzyl bromide (77 mg, 0.32 mmol) in 0.2 ml acetonitrile (a few drops of DMF were added for solubility) were stirred overnight at room temperature. The mixture was diluted with ether, the solid filtered, washed with ether and dried to give 112 mg of product. LC/MS (ES+) m/e 333[M+1]

### Example 15

### Preparation of 1-(3,4-dichlorobenyl)-3-[2-(3,4-dichlorophenyl)-2-oxoethyl]-3H-imidazol-1-ium; bromide

Following the above procedure except substituting 1-benzyl-2-methyl-1H-imidazole with 1-(3,4-dichloro-phenyl)-2-(2-methylimidazole-1-yl)-ethanone. 83 mg of the captioned product was obtained. LC/MS (ES+) m/e 416[M+1]

### Example 16

### Preparation of 3-Benzyl-2-methyl-1-phenethyl-5H-imidazol-1-ium; bromide

(2-Bromoethyl)-benzene (59 mg, 0.32 mmol) and 1-benzyl-2-methyl-1H-imidazole (50 mg, 0.29 mmol) were stirred in 0.2 ml of acetonitrile (a few drops of DMF were added for solubility) overnight at room temperature. The mixture was diluted with ether, the soid filtered, washed with ether and dried to give 67 mg of product. LC/MS (ES+) m/e 278[M+1]

### Example 17

### Preparation of [(4-Chloro-3-(trifluoromethylphenyl)oxoethyl]-(dichlorophenyl-oxoethyl)-3H-imidazole-1-ium; bromide

4-Chloro-3-(trifluoromethyl)phenacyl bromide (54 mg, 0.179 mmol) and 1-(3,4-dichlorophenyl)-2-imidazol-yl-ethanone (46 mg, 0.179 mmol) were stirred in 0.2 ml of acetonitrile (a few drops of DMF were added for solubility) overnight at room temperature. The reaction mixture was diluted with ether, the solid filtered, washed with ether and dried to give 66 mg of desired product. LC/MS (ES+) m/e 478[M+1]

### Example 18

### Preparation of 3-[2-(3-Chlorophenyl)-2-oxoethyl]-1-[2-(4-chloro-3-trifluoromethylphenyl)-2-oxoethyl]-3H-imidazol-1-ium; bromide

Following the procedure in Example 18 except substituting 3,4-dichlorophenacyl bromide with 3-chlorophenacyl bromide. 55 mg of of the captioned product was obtained. LC/MS (ES+) m/e 443[M+1]

### Example 19

### Preparation of 1,3-Bis-[2-(3,4-dichlorophenyl)-2-oxoethyl]-3H-imidazol-1-ium; bromide

3,4-dichlorophenacyl bromide (1.34 g, 5 mmol) and 2-ethylimidazole were stirred in 3 ml of DMF at 0° C. for 1 hour, then the reaction was warmed up to room temperature and stirred overnight. The mixture was poured into water, extracted with ethyl acetate. 1-(3,4-Dichloro-phenyl)-2-(2-ethylimidazole-1-yl)-ethanone (550 mg) was obtained after the solvent was removed under reduced pressure. 1-(3,4-Dichloro-phenyl)-2-(2-ethylimidazole-1-yl)-ethanone (100 mg, 0.353 mmol) and 3,4-dichlorophenacyl bromide were stirred in 0.5 ml of acetonitrile at room temperature overnight. The reaction mixture was diluted in ether, the solid filtered, washed with ether and dried to give 155 mg of desired product. LC/MS (ES+) m/e 472[M+1]

### Example 20

### Preparation of 1-[2-(3-Chlorophenyl)-2-oxoethyl]-3-[2-(3,4-dichloro-phenyl)-2-oxoethyl-3H-imidazol-1-ium; bromide

Following the above procedure except substituting 3,4-dichlorophenacyl bromide with 3-chlorophenacyl bromide in the second step. 110 mg of product was obtained. LC/MS (ES+) m/e 438[M+1]

### Example 21

### Preparation of 1,3-Bis-[2-(3,4-dichlorophenyl)-2-oxoethyl]-3H-benzoimidazol-1-ium; bromide

Benzimidazole (100 mg, 0.846 mmol) and 3,4-dichlorophenacyl bromide (227 mg, 0.846 mmol) were stirred overnight in 1 ml acetonitrile (a few drops of DMF were added for solubility). This mixture was diluted with ether, the solid filtered, washed with ether and dried to provide 95 mg of desired product. LC/MS (ES+) m/e 494[M+1]

### Example 22

### Preparation of 4-Chloro-3-(triuoromethyl)phenacyl bromide

4-Chloro-3-(trifluoromethyl)benzylaldhyde (3.0 g, 14.4 mmol) in diethyl ether was cooled to −78° C. Methylmagnesium chloride (3.0M solution in THF, 29 mmol) was added dropwise. The reaction was warmed up slowly to −20° C. TLC showed the reaction was complete. It was quenched with saturated amonium chloride solution. The aqueous layer was extracted with ethyl acetate, organic layer wre combined and washed with water, brine, and dried with magnesium sulfate. This gave 1-(4-chloro-3-trifluoromethylphenyl)ethanol (3.2 g, 99%) after the solvent was removed under reduced pressure. 1-(4-Chloro-4-trifluoromethyl-phenyl)ethanol was then oxidized by swern oxidation (2.5 eq. DMSO, 1.2 eq. oxyal chloride, 5 eq. triethylamine, 60 ml of dichloromethane, −78° C.). The resulting 1-(3-chloro-4-trifluoromethylphenyl)ethanone (3 g, 95% yield) was brominated according to a literature procedure (Horng-Chih, Huang et al., J. Med. Chem.; 1996, Vol. 39, 253-266). 4-chloro-3-(trifluoromethyl)-phenacyl bromide was obtained in 70% yield. LC/MS (ES+) m/e 303[M+1]

### Example 23

General procedure of preparing: 1-(3-fluorophenyl)-2-imidazol-1-yl-ethanone; 2-imidazol-1-yl-1-(3-methoxy-phenyl)-ethanone; 1-(3,4-dichlorophenyl)-2-(2-ethylimidazole-1-yl)-ethanone; and 1-(3,4-difluorophenyl)-2-imidazol-1-yl-ethanone

Imidazole (100 mg) and 1.0 eq. of the corresponding R-phenacyl bromide were stirred in 0.5 ml of acetonitrile (a few drops of DMF were added for solubility if necessary) overnight at room temperature. The reaction was diluted with ether, the solid filtered, washed and dried to provide product in high yield. (85-95%)

### Example 24

### 13-Bis-[2-(3,4-dichlorophenyl)-2-oxoethyl]-3H-imidazol-1-ium; bromide

The title compound was prepared following the general procedure described above. ¹H NMR (400 MHz, DMSO-d₆): 9.07 (s, 1H); 8.32 (d, J=2 Hz, 2H); 8.03 (dd, J=8.4, 2 Hz, 2H); 7.96 (d, J=8.4 Hz, 2H); 7.79 (s, 1H); 6.16 (s, 4H).

In addition, the following compounds were prepared using the same or similar conditions and the appropriate reactants:

13-Bis-[2-(3,4-dichlorophenyl)-2-oxoethyl]-2-methyl-3H-imidazol-1-ium; bromide: ¹H NMR (400 MHz, DMSO-d₆):8.32 (s, 2H); 8.02 (d, J=8.3 Hz, 2H); 7.97 (d, J=8.3 Hz, 2H); 7.65 (s, 2H); 6.14 (s, 4H); 3.34 (s, 3H).

3-[2-(4-Chlorophenyl)-2-oxoethyl]-1-[2-(3,4-dichlorophenyl)-2-oxoethyl]-3H-imidazol-1-ium; bromide. ¹H NMR (400 MHz, DMSO-d₆):9.04 (s, 1H); 8.32 (d, J=2 Hz, 1H); 8.09 (d, J=8.4 Hz, 2H); 8.04 (dd, J=8.4, 2 Hz, 1H); 7.96 (d, J=8.4 Hz, 1H); 7.77 (m, 4H); 6.13 (s, 4H).

1-[2-(3,4-Dichlorophenyl)-2-oxoethyl]-3-[2-(3-methoxyphenyl)-2-oxoethyl]-3H-imidazol-1-ium; bromide. ¹H NMR (400 MHz, DMSO-d₆):9.07 (s, 1H); 8.32 (d, J=2 Hz, 1H); 8.03 (dd, J=8.4, 2 Hz, 1H); 7.96 (d, J=8.4 Hz, 1H); 7.78 (m, 2H); 7.67 (d, J=7.6 Hz, 1H); 7.57 (dd, J=8.6, 2 Hz, 1H); 7.55 (d, J=1.6 Hz, 1H); 7.36 (dd, J=7.6, 2 Hz, 1H); 6.15 (s, 4H); 3.89 (s, 3H).

3-[2-(3,4-Dichlorophenyl)-2-hydroxyethyl]-1-[2-(3,4-dichlorophenyl)-2-oxoethyl]-3H-imidazol-1-ium; bromide. ¹H NMR (400 MHz, methanol-d4):8.93 (s, 1H); 8.25 (d, J=2 Hz, 1H); 8.00 (dd, J=8.3 Hz, 1H); 7.80 (d, J=8.3 Hz, 1H); 7.63 (s, 2H); 7.57 (m, 2H); 7.36 (dd, J=8.3, 2 Hz, 1H); 5.96 (s, 2H); 5.12 (dd, J=7.0, 3.1 Hz, 1H); 4.59 (dd, J=13.9, 3.2 Hz, 1H); 4.43 (dd, J=13.9, 7.0 Hz, 1H).

1,3-Bis-[2-(3-methoxyphenyl)-2-oxoethyl]-3H-imidazol-1-ium; bromide ¹H NMR (400 MHz, DMSO-d₆):9.10 (s, 1H); 7.80 (s, 2H); 7.67 (d, J=8.1 Hz, 2H); 7.57 (m, 4H); 7.36 (dd, J=8.1, 2.2 Hz, 2H); 6.1 (s, 4H); 3.87 (s, 6H).

1-[2-(3,4-Dichloro-phenyl)-2-oxoethyl]-3-(2-oxo-2-phenyl-ethyl)-3H-imidazol-1-ium; bromide. ¹H NMR (400 MHz, DMSO-d₆):9.08 (s, 1H); 8.32 (d, J=2 Hz, 1H); 8.07 (m, 2H); 8.05 (dd, J=8.4, 2 Hz, 1H); 7.96 (d, J=8.4 Hz, 1H); 7.79 (m, 3H); 7.66 (t, J=7.9 Hz, 2H); 6.1 (s, 4H).

1,3-Bis-[2-(3-chlorophenyl)-2-oxoethyl]-3H-imidazol-1-ium; bromide. ¹H NMR (400 MHz, DMSO-d₆):9.06 (s, 1H); 8.11 (d, J=1.6 Hz, 2H); 8.03 (d, J=7.9 Hz, 2H); 7.86 (dd, J=7.9, 1.4 Hz, 2H); 7.78 (d, J=1.3 Hz, 2H); 7.70 (t, J=7.9 Hz, 2H); 6.16 (s, 4H).

1-[2-(3-Chlorophenyl)-2-oxoethyl]-3-[2-(3-fluorophenyl)-2-oxoethyl]-3H-imidazol-1-ium; bromide. ¹H NMR (400 MHz, DMSO-d₆):9.07 (s, 1H); 8.11 (t, J=1.7 Hz, 1H); 8.04 (d, J=7.8 Hz, 1H); 7.94 (d, J=7.8 Hz, 2H); 7.88 (m, 2H); 7.78 (t, J=1.8 Hz, 2H); 7.70 (m, 3H); 6.15 (s, 4H).

1-[2-(3-Chlorophenyl)-2-oxoethyl]-3-(2-oxo-2-phenylethyl)-3H-imidazol-1-ium; bromide. ¹H NMR (400 MHz, DMSO-d₆):9.07 (s, 1H); 8.11 (m, 4H); 7.87 (dd, J=8.0, 1.2 Hz, 1H); 7.79 (m, 3H); 7.68 (m, 3H); 6.15 (s, 4H).

1-[2-(3-Chlorophenyl)-2-oxoethyl]-3-[2-(3-methoxyphenyl)-2-oxoethyl]-3H-imidazol-1-ium; bromide. ¹H NMR (400 MHz, DMSO-d₆):9.08 (s, 1H); 8.11 (t, J=1.8 Hz, 1H); 8.03 (d, J=8.0 Hz, 1H); 7.87 (d, J=8.0 Hz, 1H); 7.79 (dd, J=6.5, 1.6 Hz, 2H); 7.70 (m, 2H); 7.56 (m, 2H); 7.36 (dd, J=8.0, 2.3 Hz, 1H); 6.16 (s, 4H); 3.87 (s, 3H).

1-[2-(3-Chlorophenyl)-2-oxoethyl]-3-[2-(3,4-difluorophenyl)-2-oxoethyl]-3H-imidazol-1′-ium; bromide. ¹H NMR (400 MHz, DMSO-d₆):9.06 (s, 1H); 8.17 (m, 1H); 8.11 (t, J=1.8 Hz, 1H); 8.02 (m, 2H); 7.87 (d, J=8.0 Hz, 1H); 7.76 (m, 3H); 7.71 (m, 1H); 6.15 (s, 2H); 6.13 (s, 2H).

3-[2-(3-Chlorophenyl)-2-oxoethyl]-1-[2-(3,4-dichlorophenyl)-2-oxoethyl]-3H-imidazol-1-ium; bromide. ¹H NMR (400 MHz, DMSO-d₆):9.05 (s, 1H); 8.31 (d, J=1.8 Hz, 1H); 8.11 (d, J=1.8 Hz, 1H); 8.02 (d, J=8.0 Hz, 2H); 7.96 (d, J=8.0 Hz, 1H); 7.86 (d, J=7.8 Hz, 1H); 7.78 (s, 2H); 7.70 (t, J=7.8 Hz, 1H); 6.15 (s, 2H); 6.13 (s, 2H).

3-[2-(3-Chlorophenyl)-2-oxoethyl]-1-[2-(3,4-dichlorophenyl)-2-oxoethyl]-2-isopropyl-3H-imidazol-1-ium; bromide. ¹HNMR (400 MHz, DMSO-d₆): 8.37 (d, J=1.8 Hz, 1H); 8.12 (s, 1H); 8.04 (dd, J=8.4, 1.8 Hz, 2H); 7.97 (d, J=8.4 Hz, 1H); 7.87 (d, J=8.0 Hz, 1H); 7.70 (t, J=8.0 Hz, 1H); 7.66 (s, 2H); 6.20 (s, 4H); 3.55 (sp, J=7.2 Hz, 1H); 1.23 (d, J=7.2 Hz, 6H).

1,3-Bis-[2-(3,4-dichlorophenyl)-2-oxoethyl]-2-isopropyl-3H-imidazol-1-ium; bromide. ¹H NMR (400 MHz, DMSO-d₆): 8.37 (d, J=1.8 Hz, 2H); 8.05 (dd, J=8.4, 1.8 Hz, 2H); 7.96 (d, J=8.4 Hz, 2H); 7.67 (s, 2H); 6.21 (s, 4H); 3.55 (sp, J=7.2 Hz, 1H); 1.24 (d, J=7.2 Hz, 611).

3-[2-(4-Chloro-3-nitrophenyl)-2-oxoethyl]-1-[2-(3,4-dichlorophenyl)-2-oxoethyl]-3H-imidazol-1-ium; bromide. ¹H NMR (400 MHz, DMSO-d₆):9.05 (s, 1H); 8.73 (d, J=2.0 Hz, 1H); 8.32 (m, 2H); 8.10 (d, J=8.4 Hz, 1H); 8.03 (dd, J=8.4, 2.0 Hz, 1H); 7.96 (d, J=8.4 Hz, 1H); 7.77 (s, 2H); 6.15 (s, 2H); 6.13 (s, 2H).

Biological Assay

### Example A

Calcium mobilization assay. CXCR3 Ca²⁺ mobilization studies were carried out using Fluo 3-loaded RBL 2H₃CXCR3 and a microtiter plate-based assay using FLIPR (Molecular Devices, Sunnyvale, Calif.). Briefly, cells (about 80% confluent) were harvested and plated in 96 well black wall/clear bottom plates (Packard view plate) at approximately 40,000 cells/well and grown in the incubator for 18-24 hr. On the day of assay the media was aspirated and replaced with 100 μl Earls Mimimal Essential Media with Earls salts containing L-glutamine, 0.1% BSA, 4 μM Fluo-3 acetoxymethyl ester (Fluo-3 AM, Molecular Probes, Eugene, Oreg., USA) and 1.5 mM sulfinpyrazone. Plates were incubated for 60 min at 37° C., media was aspirated and replaced with the same media without Fluo-3 AM, and incubated for 10 min at 37° C. Cells were washed 3 times and incubated at 37° C. in 100 μl assay buffer (120 mM NaCl, 4.6 mM KCl, 1.03 mM KH₂ PO₄, 25 mM NaHCO₃, 1.0 mM CaCl₂, 11 mM glucose, 20 mM HEPES (pH 7.4) with 1.5 mM sulfinpyrazone. Plates were placed into FLIPR for analysis as described previously (Saran et al., Identification, molecular cloning, expression and characterization of a cysteinyl leukotriene receptor, Molecular Pharm., 56, 657-773, 1999). The maximal change in fluorescence after agonist addition was quantitated. The percent of maximal IP-10-induced Ca²⁺ mobilization was determined for each concentration of antagonist and the IC₅₀, defined as the concentration of test compound that inhibits 50% of the maximal response induced by 3.3 nM IP-10. For agonist potency the EC₅₀ is defined as the concentration that produces 50% of the maximal IP-10-induced response.

1. a compound of formula (I) 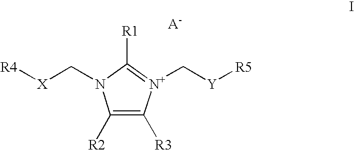
wherein:
R₁, R₂ and R₃ are the same or different and are hydrogen, C₁-C₆ alkyl, aryl, fused aryl, or heteroaryl; or are substituted aryl, fused aryl or heteroaryl;
X and Y are the same or different and are —CH₂, —C═O, —CHOH, —C═S, or —C═NR₆;
R₄ and R₅ are the same or different and are aryl, substituted aryl, heteroaryl, or substituted heteroaryl; and
R₆ is aryl, C₁-C₆ alkyl, OH, C₁-C₆ alkoxy, or aryloxy.
